(19) United States
(12) Patent Application Publication
(10) Pub. No.: US 2026/0117197 A1
(43) Pub. Date: Apr. 30, 2026

(54) METHOD FOR PRESERVING AND TRANSPORTING CELL AGGREGATE

(71) Applicant: KANEKA CORPORATION, Osaka-shi, Osaka (JP)

(72) Inventors: Nobuyoshi UMEDA, Kobe-shi (JP); Rio ADACHI MASAYASU, Kobe-shi (JP); Yuzuha ICHIKAWA, Kobe-shi (JP)

(73) Assignee: KANEKA CORPORATION, Osaka-shi, Osaka (JP)

(21) Appl. No.: 19/118,530

(22) PCT Filed: Oct. 6, 2023
(86) PCT No.: PCT/JP2023/036518

§ 371 Date: Apr. 4, 2025

(30) Foreign Application Priority Data
Oct. 7, 2022 (JP) 2022-162224

(51) Int. Cl. C12N 5/00

(52) U.S. Cl. CPC C12N 5/522

(57) ABSTRACT

Disclosed is a highly versatile method for preserving and transporting a cell aggregate capable of preserving and transporting a cell aggregate while maintaining the form of the cell aggregate. The method of preserving the cell aggregate includes a step of filling a filling vessel with a cell aggregate suspension such that the ratio of the volume of the cell aggregate suspension to the capacity of the filling vessel is 60% or higher, and a step of preserving the filling vessel in a non-frozen state for one hour or longer.

### TECHNICAL FIELD

The present invention relates to a method of preserving and transporting a cell aggregate suspension.

### BACKGROUND ART

In recent years, attempts have been made to use a cell aggregate preparation obtained by processing cells not into single cells but into a cell aggregate (e.g., spheroid, organoid, or the like) having a three-dimensional structure with the aim of improving the post-administration take rate and enhancing therapeutic efficacy in local administration of therapeutic cells used in regenerative/cell therapy. Conventionally, the regenerative/cell therapy has been performed in medical institutions having advanced equipment and technology in many cases, and therefore single cells have generally been prepared into a cell aggregate for use in medical institutions, laboratories adjacent to medical institutions, or the like, and then administered to patients.

However, to disseminate treatment using a cell aggregate, it is necessary to develop a ready-to-use cell aggregate preparation such that small medical institutions not having equipment or technicians to produce and prepare the cell aggregate can also provide the treatment using a cell aggregate.

More specifically, it has been demanded to develop a technology of preparing and preserving a cell aggregate in facilities of pharmaceutical companies or cell processing companies and transport the cell aggregate from the facilities to medical institutions while maintaining the quality and the form of the cell aggregate. Single-cell preparations are generally cryopreserved in the facilities, and then transported in a frozen state from the facilities to medical institutions. However, it is difficult to cryopreserve cell aggregate preparations without causing excessive damage to the cell aggregate with current technologies. Thus, it is assumed that the cell aggregate preparations have been demanded to be preserved and transported in a non-frozen state.

To that end, Patent Document 1 discloses a material of a vessel, the shape of a syringe, and the composition of a cell suspension, for example, suitable for preserving and transporting single-cell preparations while maintaining a high survival rate in a non-frozen state, but does not disclose advantageous effects of cell aggregate preparations. Patent Document 2 discloses a method of encapsulating a cell aggregate in hydrogel and sealing the same in a vessel for storage and transportation in a non-frozen state. The method of transporting a cell aggregate using hydrogel as in Patent Document 2 requires a step of removing the hydrogel in medical institutions after transportation, which is complicated, so that the transportation method does not lead to the widespread use of the treatment.

### CITATION LIST

### Patent Literature

Patent Literature 1: JP 5899246 B
Patent Literature 2: JP 2021-511078 T

### SUMMARY OF INVENTION

### Technical Problem

As described above, the development of the ready-to-use cell aggregate preparation requires a method of preserving and transporting the cell aggregate preparation while maintaining the quality and the shape in a non-frozen state. However, according to examinations of the present inventors, it has been clarified that, when the preservation and transportation method suitable for the single-cell preparation as in Patent Literature 1 is applied as it is to the preservation and the transportation of the cell aggregate preparation, the survival rate of the cells can be maintained but another problem, such as the collapse of the cell aggregate due to vibrations or shocks during preservation and transportation, arises. Thus, an object of the present invention is to provide a highly versatile method of preserving and transporting a cell aggregate capable of preserving and transporting a cell aggregate while maintaining the form of the cell aggregate.

### Solution to Problem

The present inventors have conducted extensive examinations to solve the above-described problems, and, as a result, have found a preservation and transportation method capable of maintaining the form of a cell aggregate even when the cell aggregate is subjected to vibrations or shocks during preservation and transportation by filling a vessel with a cell aggregate suspension under specific conditions, and then preserving and transporting the same in a non-frozen state. Thus, the present invention has the following (1) to (10) characteristics:
(1) A method of preserving a cell aggregate containing at least one of a pluripotent stem cell, a pluripotent stem cell-derived differentiated cell, and a somatic stem cell, the method including:
a step of filling a filling vessel with a cell aggregate suspension such that the ratio of the volume of the cell aggregate suspension to the capacity of the filling vessel is 60% or higher (filling step); and
a step of preserving the filling vessel in a non-frozen state for one hour or longer (preservation step).
(2) The method according to (1), in which the ratio of the volume of the cell aggregate suspension to the capacity of the filling vessel is 70% or higher.
(3) The method according to (1) or (2), in which the ratio of the volume of the cell aggregate suspension to the capacity of the filling vessel is 80% or higher.
(4) The method according to any one of (1) to (3), in which the preservation is transportation or comprises transportation.
(5) The method according to any one of (1) to (4), in which the cell aggregate suspension is obtained by suspending a cell aggregate in a non-freezing stock solution.
(6) The method according to any one of (1) to (5), in which the non-freezing stock solution is a solution containing a sodium salt.
(7) The method according to (6), in which the non-freezing stock solution is a Ringer's solution or an aqueous solution prepared from a Ringer's solution.
(8) The method according to any one of (1) to (7), further including a packing step of packing the filling vessel filled with the cell aggregate suspension in a constant temperature vessel together with a cold insulator, in which the filling vessel is given with cell information and/or information on a subject to be administered.
(9) The method according to any one of (1) to (8) above, in which the cell aggregate to be subjected to the preservation step has an average diameter of 600 μm or lower.
(10) The method according to any one of (1) to (9) above, wherein the filling vessel is preserved at 15° C. or lower in the preservation step.

This specification encompasses the contents disclosed in Japanese Patent Application No. 2022-162224, on which the priority of the present application is based.

### Advantageous Effects of Invention

The present invention can provide a method of preservation and transportation capable of maintaining the form of a cell aggregate even when the cell aggregate is subjected to vibrations or shocks during preservation and transportation by filling a vessel with a cell aggregate suspension under specific conditions, and then preserving and transporting the same in a non-frozen state.

### BRIEF DESCRIPTION OF DRAWING

FIG. 1 shows photographs showing the forms of spheroids after an adipose-derived MSC spheroid suspension was filled into vials using various filling methods, followed by non-frozen preservation for 24 hours under vibrational conditions.

FIG. 2 shows photographs of the forms of spheroids after a human iPS cell spheroid suspension was filled into vials using various filling methods, followed by non-frozen preservation for 24 hours under vibrational conditions.

### DESCRIPTION OF EMBODIMENTS

### 1. Method of Producing Cell Aggregate Composition

### 1.1 Definition of Terms

The following terms used in this specification are defined.

### <<Cells>>

A “pluripotent stem cell” as a subject matter of the invention in this specification refers to a cell having pluripotent capacity (pluripotency) to differentiate into all types of cells constituting a living body and being capable of permanently continuing proliferation with the pluripotency maintained in vitro culture under adequate conditions. More specifically, pluripotency means an ability to differentiate into germ layers constituting an individual (for vertebrates, three germ layers: ectoderm, mesoderm, and endoderm). Examples of such cells include embryonic stem cells (ES cells), embryonic germ cells (EG cells), germline stem cells (GS cells), and induced pluripotent stem cells (iPS cells).

The “ES cell” is a pluripotent stem cell prepared from an early embryo. The “EG cell” is a pluripotent stem cell prepared from a fetal primordial germ cell (Shamblott M. J. et al., 1998, Proc. Natl. Acad. Sci., U.S.A., 95:13726-13731). The “GS cell” is a pluripotent stem cell prepared from a testicular cell (Conrad S., 2008, Nature, 456:344-349). The “iPS cell” refers to a pluripotent stem cell that has been reprogrammed by introducing a few genes encoding initialization factors into a differentiated somatic cell to bring the somatic cell into an undifferentiated state.

The pluripotent stem cells in this specification may be cells derived from any multicellular organism and having the above-described characteristics. The pluripotent stem cells are preferably pluripotent stem cells derived from an animal, and more preferably pluripotent stem cells derived from a mammal. Examples thereof include a rodent, such as a mouse, a rat, a hamster and a guinea pig, a domestic or pet animal, such as a dog, a cat, a rabbit, a bovine, a horse, sheep and a goat, a primate, such as a human, a rhesus monkey, a gorilla, a chimpanzee, and the like. Pluripotent stem cells derived from a human are particularly preferable.

The pluripotent stem cells used in this specification include naive pluripotent stem cells and primed pluripotent stem cells. By definition, the naive pluripotent stem cell is in a state with pluripotency close to that found in the preimplantation inner cell mass, and the primed pluripotent stem cell is in a state with pluripotency close to that found in the postimplantation epiblast. As compared with the naive pluripotent stem cells, the primed pluripotent stem cells are characterized by less frequent contribution to ontogenesis, X-chromosome transcription activity found only for one chromatid, and high-level transcription-suppressive histone modification. A marker gene for the primed pluripotent stem cells is OTX2, and marker genes for the naive pluripotent stem cells are REX1 and the KLF family. The primed pluripotent stem cells form flat colonies, and the naive pluripotent stem cells form dome-shaped colonies. In particular, it is preferable to use the primed pluripotent stem cells for the pluripotent stem cells used in this specification.

Commercially available or donated cells or newly prepared cells may be used as the pluripotent stem cells used in this specification. In use for the invention of this specification, the pluripotent stem cells are preferably, but not limited to, iPS cells or ES cells.

When a commercially available product or a research strain is used as the iPS cells in this specification, examples of the products and the strains that can be used include, but are not limited to, 253G1 strain, 253G4 strain, 201B6 strain, 201B7 strain, 409B2 strain, 454E2 strain, 606A1 strain, 610B1 strain, 648A1 strain, HiPS-RIKEN-1A strain, HiPS-RIKEN-2A strain, HiPS-RIKEN-12A strain, Nips-B2 strain, TkDN4-M strain, TkDA3-1 strain, TkDA3-2 strain, TkDA3-4 strain, TkDA3-5 strain, TkDA3-9 strain, TkDA3-20 strain, hiPSC 38-2 strain, MSC-iPSC1 strain, BJ-iPSC1 strain, RPChiPS771-2, WTC-11 strain, 1231A3 strain, 1383D2 strain, 1383D6 strain, 1210B2 strain, 1201C1 strain, 1205B2 strain, and the like.

When iPS cells for clinical use are used as the iPS cells in this specification, examples of strains include, but are not limited to, QHJI01s01 strain, QHJI01s04 strain, QHJI14s03 strain, QHJI14s04 strain, Ff-114s03 strain, Ff-114s04 strain, YZWI strain, and the like.

When the iPS cells used in this specification are newly prepared cells or self-produced cells, combinations of genes for initialization factors to be introduced may include, but are not limited to, a combination of the OCT3/4 gene, the KLF4 gene, the SOX2 gene, and the c-Myc gene (Yu J, et al. 2007, Science, 318:1917-20) and a combination of the OCT3/4 gene, the SOX2 gene, the LIN28 gene, and the Nanog gene (Takahashi K, et al. 2007, Cell, 131:861-72).

The introduction form of these genes into cells is not particularly limited, and may be, for example, gene introduction using a plasmid, such as an episomal vector, introduction of a synthetic RNA, or introduction as a protein. Further, iPS cells produced by methods using a Sendai virus vector, microRNA or RNA, a low molecular weight compound, and the like may be acceptable. In addition, universal iPS cells from which an HLA gene has been edited and removed to suppress immune rejection may also be acceptable.

When a commercially available product is used as the ES cells in this specification, the commercially available products may include, but are not limited to, KhES-1 strain, KhEs-2 strain, KhEs-3 strain, KhEs-4 strain, KhEs-5 strain, SEES1 strain, SEES2 strain, SEES3 strain, SEES-4 strain, SEES-5 strain, SEES-6 strain, SEES-7 strain, HUES8 strain, CyT49 strain, H1 strain, H9 strain, HS-181 strain, and the like.

The “pluripotent stem cell-derived differentiated cell” as a subject matter of the invention in this specification is a cell differentiated from the above-described pluripotent stem cell into any tissue cell constituting a living body. The differentiation as used herein may be the unintentional deviation from the undifferentiated state to be, for example, a stem cell of a germ layer lineage or the like or may be the intentional induction into any tissue cell. A differentiation induction method into any tissue cell is not particularly limited insofar as the method is capable of inducing tissue cells.

Examples of the pluripotent stem cell-derived differentiated cells include, for example, but are not limited to, a cardiac cell, a neural cell, a retinal cell, a liver cell, a pancreatic islet cell, and the like. In addition to these cells, a progenitor cell, such as a cardiac progenitor cell, or a stem cell may be acceptable, and the maturity of the differentiated tissue cells is not particularly limited and optional. In this specification, the pluripotent stem cell-derived differentiated cell is sometimes referred to as a “differentiated cell”, a “tissue cell”, and the like.

The “somatic stem cell” as a subject matter of the invention in this specification is a stem cell present in a living body and refers to a stem cell that can be differentiated into a limited number of cells. Examples of the somatic stem cell include, for example, a mesenchymal stem cell, a neural stem cell, an intestinal epithelial stem cell, a hair follicle stem cell, a mammary stem cell, and a pigment stem cell. When the somatic stem cell is used, the tissue from which the cell is derived is not particularly limited. For example, in the case of the somatic stem cell present in various tissues, such as the mesenchymal stem cell, cells derived from any tissue may be used or cells derived from a plurality of different tissues may be used in combination.

In this specification, the “mesenchymal stem cell (MSC)” refers to a cell that meets the following definition and refers to a somatic stem cell having differentiation potential capable of differentiating into one or more types of cells belonging to a mesodermal tissue. The mesenchymal stem cell in this specification includes both a mesenchymal stem cell obtained from any tissue and a mesenchymal stem cell prepared in vitro.
i) Exhibiting adhesion to plastics under a culture condition on a standard medium. The standard medium is a medium in which serum, a serum replacement reagent, or a growth factor is added to a basal medium (e.g., aMEM medium).
ii) Surface antigens CD73 and CD90 are positive and CD34 and 45 are negative.

In this specification, a “therapeutic cell” means a cell administered to a subject for the purpose of cell therapy.

<<Cell aggregate>>

In this specification, the “cell aggregate” is a massive cell population formed through cell aggregation, and is also referred to as a spheroid. A cell aggregate is substantially spherical in general. The cell aggregate in this specification is preferably produced in suspension culture. In this case, the suspension culture may be performed either in a static state or in a flow state. Examples of the flow state include, but are not limited to, rotational culture, rocking culture, stirring culture, and combinations thereof.

Cells constituting a cell aggregate are not particularly limited insofar as they are one or more types of the above-described cells. For example, the cells constituting a cell aggregate are sometimes only pluripotent stem cells, sometimes only a specific species of pluripotent stem cell-derived differentiated cells, sometimes a plurality of types of pluripotent stem cell-derived differentiated cells, or sometimes only somatic stem cells, although this is not a particular limitation.

For example, a cell aggregate composed of pluripotent stem cells, such as human pluripotent stem cells or human embryonic stem cells, include cells expressing a pluripotent stem cell marker and/or being positive for a pluripotent stem cell marker. The pluripotent stem cell markers are gene markers specifically or excessively expressed in pluripotent stem cells, and examples thereof can include Alkaline Phosphatase, Nanog, OCT4, SOX2, TRA-1-60, c-Myc, KLF4, LIN28, SSEA-4, SSEA-1, and the like.

For example, a cell aggregate composed of pluripotent stem cell-derived neural cells includes cells expressing a neural marker and/or being positive for a neural marker. The neural markers are gene markers specifically or excessively expressed in neural cells, and examples thereof can include PAX6, SOX1, CHX10, and the like.

For example, a cell aggregate composed of pluripotent stem cell-derived pancreatic islet cells includes cells expressing an islet marker and/or being positive for a pancreatic islet marker. The pancreatic islet markers are gene markers specifically or excessively expressed in pancreatic islet cells, and examples thereof can include PDX1, NKX6.1, and the like.

For example, a cell aggregate composed of pluripotent stem cell-derived cardiac cells includes cells expressing a cardiac marker and/or being positive for a cardiac marker. The cardiac markers are gene markers expressed or excessively expressed in cardiac cells, examples thereof can include cTnT, ML2Cv, Actinin, and the like.

For example, a cell aggregate composed of somatic stem cells, such as human mesenchymal stem cells, includes cells expressing positive markers characteristic of mesenchymal stem cells and contains cells not expressing negative markers characteristic of mesenchymal stem cells. The positive markers of mesenchymal stem cells are cell surface antigen markers expressed in mesenchymal stem cells, and examples thereof can include CD73, CD90, CD105, and the like. The negative markers of mesenchymal stem cells are cell surface antigen markers not expressed in mesenchymal stem cells, and examples thereof can include CD34, CD45, and the like.

The pluripotent stem cell markers, the neural markers, the pancreatic islet markers, the cardiac markers, the mesenchymal stem cell markers, and the like can be detected with any detection method in the art. Examples of methods for detecting cell markers include, but are not limited to, flow cytometry. In a case where a fluorescence-labeled antibody is used as a detection reagent in flow cytometry, when a cell emitting more intense fluorescence than a negative control (isotype control) is detected, the cell is determined to be “positive” for the marker.

The proportion of cells positive for a fluorescence-labeled antibody as analyzed by flow cytometry is sometimes referred to as the “positive rate”. Any antibody known in the art can be used as the fluorescence-labeled antibodies, and examples thereof include, but are not limited to, antibodies labeled with fluorescein isothiocyanate (FITC), phycoerythrin (PE), allophycocyanin (APC), and the like.

When cells constituting a cell aggregate are pluripotent stem cells, the positive rate for the pluripotent stem cell marker can be set to preferably 80% or higher, more preferably 90% or higher, more preferably 91% or higher, more preferably 92% or higher, more preferably 93% or higher, more preferably 94% or higher, more preferably 95% or higher, more preferably 96% or higher, more preferably 97% or higher, more preferably 98% or higher, more preferably 99% or higher, and more preferably 100% or lower. Cell aggregates in which the percentage of cells expressing the pluripotent stem cell marker and/or being positive for the pluripotent stem cell marker falls within the ranges above are highly undifferentiated and highly homogeneous cell populations.

When cells constituting a cell aggregate are neural cells, the positive rate for the neural marker can be set to preferably 80% or higher, more preferably 90% or higher, more preferably 91% or higher, more preferably 92% or higher, more preferably 93% or higher, more preferably 94% or higher, more preferably 95% or higher, more preferably 96% or higher, more preferably 97% or higher, more preferably 98% or higher, more preferably 99% or higher, and more preferably 100% or lower. Cell aggregates in which the percentage of cells expressing the neural marker and/or being positive for the neural marker falls within the ranges above are considered to be cell aggregates that are highly homogeneous and safer and have high therapeutic efficacy.

When cells constituting a cell aggregate are pancreatic islet cells, the positive rate for the pancreatic islet marker can be set to preferably 80% or higher, more preferably 90% or higher, more preferably 91% or higher, more preferably 92% or higher, more preferably 93% or higher, more preferably 94% or higher, more preferably 95% or higher, more preferably 96% or higher, more preferably 97% or higher, more preferably 98% or higher, more preferably 99% or higher, and more preferably 100% or lower. Cell aggregates in which the percentage of cells expressing the pancreatic islet marker and/or being positive for the pancreatic islet marker falls within the ranges above are considered to be cell aggregates that are highly homogeneous and safer and have high therapeutic efficacy.

When cells constituting a cell aggregate are cardiac cells, the positive rate for the cardiac marker can be set to preferably 80% or higher, more preferably 90% or higher, more preferably 91% or higher, more preferably 92% or higher, more preferably 93% or higher, more preferably 94% or higher, more preferably 95% or higher, more preferably 96% or higher, more preferably 97% or higher, more preferably 98% or higher, more preferably 99% or higher, and more preferably 100% or lower. Cell aggregates in which the percentage of cells expressing the cardiac marker and/or being positive for the cardiac marker falls within the ranges above are considered to be cell aggregates that are highly homogeneous and safer and have high therapeutic efficacy.

When cells constituting a cell aggregate are mesenchymal stem cells, the positive rate of the positive marker of a mesenchymal stem cell can be set to preferably 80% or higher, more preferably 90% or higher, more preferably 91% or higher, more preferably 92% or higher, more preferably 93% or higher, more preferably 94% or higher, more preferably 95% or higher, more preferably 96% or higher, more preferably 97% or higher, more preferably 98% or higher, more preferably 99% or higher, and more preferably 100% or lower. The positive rate of the negative marker of a mesenchymal stem cell can be set to preferably 20% or lower, more preferably 10% or lower, more preferably 9% or lower, more preferably 8% or lower, more preferably 7% or lower, more preferably 6% or lower, more preferably 5% or lower, more preferably 4% or lower, more preferably 3% or lower, more preferably 2% or lower, more preferably 1% or lower, and more preferably 0%.

### <<Cell Aggregate Suspension>>

In this specification, a “cell aggregate suspension” refers to a population of any cell aggregate suspended in a non-freezing stock solution, and may be a cell aggregate preparation administered to a living body or may be a cell aggregate in an intermediate stage in which the cell aggregate is subjected to further treatment (culture, differentiation, replacement of a solution in which the cell aggregate is suspended, addition of components, and the like) for the preparation of a cell aggregate preparation. The non-freezing stock solution in the present invention is itself pharmacologically acceptable and can be administered directly to a living body.

From the viewpoint of reducing damage to cells, the “cell aggregate suspension” preferably has a low temperature, and, for example, is preferably in a 15° C. or lower non-frozen state.

The “cell aggregate suspension” may be in a state where it is divided into any amount and preserved. Examples of vessels for dividing and preserving the cell aggregate suspension include a vial and a bag. The cell aggregate suspension in the state where the cell aggregate is suspended may be in the form of a liquid, a viscous liquid, or a gel. The cells in the cell aggregate suspension are mainly in the cell aggregate state, but sometimes in a state where unicellular cells are mixed.

In this specification, the “cell aggregate suspension” is sometimes referred to as an “aggregate suspension”, and the division of cells into a plurality of vessels is sometimes referred to as “filling” or “dispensing”. The “vessel” filled with the cells is sometimes referred to as a “filling vessel”. In this specification, a method of preserving the cell aggregate suspension sometimes simply refers to transporting the cell aggregate.

### <<Non-Freezing Stock Solution>>

In this specification, a “non-freezing stock solution” refers to a solution that allows non-frozen preservation of cells. The non-freezing stock solution of the present invention allows non-frozen preservation of cells, and is a preservation solution containing a sodium salt, a potassium salt, and a calcium salt. For example, the non-freezing stock solution is physiological saline, a Ringer's solution, a lactated Ringer's solution, a bicarbonated Ringer's solution, an acetated Ringer's solution, or a solution in which additional components are appropriately added to physiological saline, Ringer-based solutions, or the like. In this specification, the “non-freezing stock solution” is often referred to as a “stock liquid”, a “stock solution”, or the like. As the “non-freezing stock solution” in this specification, solutions that are not originally intended for preservation under non-freezing conditions (e.g., stock solutions for freezing or solutions used for other purposes) can be used insofar non-freezing preservation of cells can be achieved.

In this specification, the “solution containing a sodium salt (hereinafter often simply referred to as “solution” in this specification)” refers to any aqueous solution containing a sodium salt. In addition to a sodium salt, a potassium salt and a calcium salt may be contained. The osmotic pressure, ion composition, and pH are not particularly limited. Specific examples of the solution include solutions used as injections, such as extracellular fluid replacement solutions, e.g., physiological saline, a Ringer's solution, and the like, hypotonic electrolyte solutions, peripheral intravenous nutrition injections, high-calorie injections, plasma substitute volume expanders, and the like.

A “sodium salt” refers to a compound containing a sodium ion as a cation and a hydrate thereof. Specific examples thereof include sodium chloride, sodium carbonate, sodium bicarbonate, sodium sulfate, sodium acetate, sodium lactate, sodium phosphate monobasic, sodium phosphate dibasic, sodium phosphate, sodium gluconate, sodium citrate, and hydrates thereof.

A “potassium salt” refers to a compound containing a potassium ion as a cation and a hydrate thereof. Specific examples thereof include potassium chloride, potassium carbonate, potassium bicarbonate, potassium acetate, potassium lactate, potassium iodide, potassium bromide, potassium sulfate, potassium gluconate, potassium citrate, and hydrates thereof.

A “calcium salt” refers to a compound containing a calcium ion as a cation and a hydrate thereof. Specific examples thereof include calcium chloride, calcium carbonate, calcium bicarbonate, calcium acetate, calcium lactate, calcium sulfate, calcium phosphate, calcium dihydrogen phosphate, calcium gluconate, calcium citrate, and hydrates thereof.

A “chloride” refers to a compound containing a chloride ion as an anion and a hydrate thereof. Specific examples thereof include sodium chloride, potassium chloride, calcium chloride, magnesium chloride, and hydrates thereof.

In this specification, a “Ringer's solution” refers to a solution containing sodium chloride, potassium chloride, and calcium chloride and having the osmotic pressure that allows cells to survive. The Ringer's solution typically includes a Ringer's basal solution, a lactated Ringer's solution, an acetated Ringer's solution, and a bicarbonated Ringer's solution. The Ringer's solution does not contain growth factors that have a significant influence on the characteristics and properties of cells, and thus is suitable as the stock solution of the present invention.

In this specification, a “Ringer's basal solution” refers to one type of the Ringer's solution, and refers to a solution in which a sodium salt, a potassium salt, and a calcium salt are all chlorides.

In this specification, a “lactated Ringer's solution” refers to a Ringer's solution containing a lactate ion as an anion. Typically, a lactate ion is contained, so that the sodium ion concentration is higher than the chloride ion concentration.

In this specification, an “acetated Ringer's solution” refers to a Ringer's solution containing an acetate ion as an anion. Typically, an acetate ion is contained, so that the sodium ion concentration is higher than the chloride ion concentration.

In this specification, a “bicarbonated Ringer's solution” refers to a Ringer's solution containing a bicarbonate ion as an anion. Typically, a bicarbonate ion is contained, so that the sodium ion concentration is higher than the chloride ion concentration.

### <<Cell Information>>

In this specification, “cell information” refers to information on the type, strain, properties, origin, provenance, and the like of therapeutic cells contained in the filling vessel and information useful for identifying cells.

### <<Subject>>

In this specification, a “subject” refers to an individual to whom a cell preparation of the present invention is applied. For example, the “subject” is a “patient” in need of treatment. The definition of the “patient” is a person who has some kind of abnormality in the body and is not in a healthy state, e.g., a person who is suffering from some kind of disease.

In this specification, a “cell preparation” means a live cell applicable to the subject or a composition containing live cells serving as the raw material of a therapeutic cell. In this specification, “subject information” refers to information on the name, age, gender, disease name, and the like of the subject who receives the administration of a therapeutic cell suspension, and means information useful for identifying the subject. The “cell information” and the “subject information” may be given in the form of a symbol or a barcode. The specific information corresponding to the given symbol or barcode can also be confirmed via an email or a database.

### <<Cold Insulator>>

In this specification, a “cold insulator” means an agent capable of keeping the “filling vessel” at a low temperature or a sealed small bag sealing the agent. For example, the “cold insulator” is a bag packed with a composition containing water and a highly absorbent resin (e.g., sodium polyacrylate) as the main component.

### <<Constant Temperature Vessel>>

In this specification, a “constant temperature vessel” means a vessel having insulating properties that enable the temperature inside the vessel to be kept in a certain temperature range for a certain period of time.

### <<Adherent Culture>>

“Adherent culture” is one of cell culture methods and refers to allowing cells to proliferate as a monolayer in principle with the cells that are made to adhere to an external matrix or the like, such as a culture vessel. The external matrix is not particularly limited, and, for example, a common plastic culture vessel used for culturing adherent cells, such as mesenchymal stem cells, is usable. To further promote cell adhesion and proliferation, the above-described culture vessel may be coated with Laminin, Vitronectin, Gelatin, Collagen, E-Cadherin chimeric antibody, or the like. Cells grown in the adherent culture form dense cell colonies. The above-described cells can usually be cultured not only by the adherent culture but also by suspension culture.

### <<Suspension Culture>>

“Suspension culture” is one of cell culture methods and refers to culturing cells in a suspension state in a liquid medium. In this specification, a “suspension state” refers to a state in which cells do not adhere to an external matrix, such as a culture vessel. In a “suspension culture method”, cells are subjected to suspension culture. According to such a method, cells are present as a cell mass formed through aggregation in a culture solution. A method of suspending cells is not particularly limited, and a flow culture in which a medium is allowed to flow using means, such as stirring, rotating, or shaking, is usable. Alternatively, the suspension culture can be performed even in a static state by the use of a culture vessel suitable for forming a spheroid, and suspension culture means are not limited. The above-described cells can usually be cultured not only by the suspension culture but also by the adherent culture.

### <<Medium and Medium Exchange>>

A “medium” used herein refers to a liquid or solid substance prepared for cell culture. In principle, a medium contains components indispensable for proliferation and/or maintenance of cells over their minimum requirements. Unless otherwise stated, a liquid medium for animal cells for use in culture of cells derived from an animal is used as the medium in this specification.

In this specification, a “basal medium” refers to a medium that serves as a base for media for various animal cells. Culture can be performed with the use of a basal medium by itself, and media specific to different cells for different purposes may be prepared with the addition of various culture additives. Examples of the basal medium used in this specification include, but are not particularly limited to, BME medium, BGJb medium, CMRL1066 medium, Glasgow MEM medium, Improved MEM Zinc Option medium, IMDM medium (Iscove's Modified Dulbecco's Medium), Medium 199 medium, Eagle MEM medium, αMEM medium, DMEM medium (Dulbecco's Modified Eagle's Medium), Ham's F10 medium, Ham's F12 medium, RPMI 1640 medium, Fischer's medium, mixed media thereof (e.g., DMEM/F12 medium (Dulbecco's Modified Eagle's Medium/Nutrient Mixture F-12 Ham)), and the like.

For DMEM/F12 medium, in particular, a medium is used which is obtained by mixing DMEM medium and Ham's F12 medium at a weight ratio preferably in the range of 60/40 or higher to 40/60 or lower, such as 58/42, 55/45, 52/48, 50/50, 48/52, 45/55, and 42/58. In addition, other media used for culture of human iPS cells and human ES cells or culture of tissue cells can be suitably used.

The medium used in the present invention is preferably a medium containing no serum, in other words, a serum-free medium.

In this specification, the “culture additive” is a substance other than serum, which is added to a medium for the purpose of culture. Specific examples of the culture additives include, but are not limited to, human platelet lysate (hPL), L-ascorbic acid, insulin, transferrin, selenium, sodium bicarbonate, growth factors, fatty acid or lipid, amino acids (e.g., non-essential amino acids), vitamins, cytokines, antioxidants, 2-mercaptoethanol, pyruvic acid, buffers, inorganic salts, antibiotics, and the like. Insulin, transferrin, and cytokines may be naturally occurring ones separated from tissue, serum, or the like of an animal (preferably a human, mouse, rat, bovine, horse, goat, or the like) or genetically engineered recombinant proteins.

Examples of the growth factors include, but are not limited to, FGF2 (Basic fibroblast growth factor-2), TGF-B1 (Transforming growth factor-β1), Activin A, IGF-1, MCP-1, IL-6, PAI, PEDF, IGFBP-2, LIF, and IGFBP-7.

Examples of the antibiotics include, but are not limited to, penicillin, streptomycin, amphotericin B, and the like. Particularly preferable growth factors when culturing a pluripotent stem cell as the culture additives of a medium used in the present invention are FGF2 and/or TGF-β1.

As the culture additive of a medium used in the present invention, the culture additive that is particularly preferable when culturing a mesenchymal stem cell is a human platelet lysate (hPL). The human platelet lysate is preferably subjected to bacterial and viral inactivation and/or sterilization treatment. As the above-described human platelet lysate, a commercially available human platelet lysate may be used. Examples thereof include, but are not limited to, Stemulate (manufactured by Cook Regentec), PLTMax (manufactured by Mill Creek Life Sciences Inc.), UltraGRO (manufactured by AventaCell BioMedical Co., Ltd.), PLUS (manufactured by Compass Biomedical), and the like.

The media used in the early stage of culturing of pluripotent stem cells and some differentiated cells preferably contain a ROCK inhibitor. Examples of the ROCK inhibitor include Y-27632. The addition of the ROCK inhibitor to the media can significantly suppress cell death in a non-adhesion state of pluripotent stem cells to substrates or other cells and/or under high shear stress.

The medium used in the present invention can contain one or more types of the culture additives. The medium to which the culture additives are added is typically the basal media, although this is not a limitation.

The culture additive in the form of, for example, a solution, derivative, salt, a mixed reagent, or the like can be added to a medium. For example, L-ascorbic acid in the form of a derivative, such as magnesium ascorbyl-2-phosphate, may be added to a medium, and selenium in the form of a selenite (such as sodium selenite) may be added to a medium. Insulin, transferrin, and selenium in the form of an ITS reagent (insulin-transferrin-selenium) can be added to a medium.

A commercially available medium to which at least one substance is selected from among L-ascorbic acid, insulin, transferrin, selenium, and sodium bicarbonate is also usable. Examples of the commercially available media to which insulin and transferrin have been added include CHO-S-SFM II (Life Technologies Japan Ltd.), Hybridoma-SFM (Life Technologies Japan Ltd.), eRDF Dry Powdered Media (Life Technologies Japan Ltd.), UltraCULTURE™ (BioWhittaker), UltraDOMA™ (BioWhittaker), UltraCHO™ (BioWhittaker), UltraMDCK™ (BioWhittaker), STEMPRO (registered trademark) hESC SFM (Life Technologies Japan Ltd.), Essential 8™ (Life Technologies Japan Ltd.), StemFit (registered trademark) AK02N (Ajinomoto Co., INC.), StemFit (registered trademark) AK03N (Ajinomoto Co., Inc.), mTeSR1 (VERITAS Corporation), TeSR2 (VERITAS Corporation), CarmyA medium (Myoridge Co., Ltd.), and the like.

In this specification, “medium exchange” refers to the supply of a medium to cells as a nutrient source for cell survival and proliferation and the removal of a medium after the cells have consumed nutrients and accumulated metabolic products. A medium exchange method is not particularly limited, and examples thereof include a batch method, a perfusion method, and the like. The batch method means replacing all, half, or any amount of a medium in a culture system with a fresh medium at any culture time. The perfusion method refers to separately supplying a medium while continuously removing a medium from a culture system to continue performing medium exchange, and the amount of a medium removed and supplied per unit time is referred to as the perfusion quantity. The perfusion of a medium may be continuously performed or intermittently performed. In the suspension culture, the medium exchange is preferably performed in the perfusion mode.

### 1-2. Construction

Each step of a method of preserving a cell aggregate of the present invention is described. As essential steps, a step of filling a cell aggregate suspension containing pluripotent stem cells, pluripotent stem cell-derived differentiated cells, or somatic stem cells and a preservation step are essentially included. A packing step of packing a filling vessel filled with the cell aggregate suspension into a constant temperature vessel together with a cold insulator may be included as necessary. Hereinafter, each step is described.

### 1-2-1. Filling Step

In the filling step of the present invention, the filling vessel is filled with the cell aggregate suspension such that the ratio of the volume of the cell aggregate suspension to the capacity of the filling vessel is a specific ratio. The filling vessel used in the present invention is not particularly limited insofar as it is a vessel for preserving a cell aggregate suspension, and is preferably a vessel that can be sealed to prevent the contents from leaking during preservation and transportation and more preferably a vessel that can maintain an aseptic state. As the form, a vial type, a bag type, a tube type, a syringe type, an ampule type, and the like are usable. Among the above, the form of a vial type is preferably used. A filling vessel manufactured for medical use and sterilized is more preferable and individually packed filling vessels are suitably used.

The vial is a vessel for preserving a liquid, and generally contains a cylindrical vessel and a cap. In general, the vial has a structure that allows the contents to be sealed with a screw-type cap.

The bag is a vessel for preserving a liquid, is generally flat in shape, and is filled with a liquid via a tube attached to the tip of the bag. In general, the bag has a structure that allows the contents to be sealed by heat sealing.

The tube is a vessel for preserving a liquid, and generally has a structure that allows the contents to be sealed by heat sealing the tip (filling port) of the tube. The syringe type is an instrument that can suck and inject liquids or gases, and generally has a structure in which a cylindrical cylinder is provided on the outside and a movable plunger is combined on the inside.

As the material of the filling vessel, there are a plastic filling vessel, a glass filling vessel, a metal filling vessel, and the like, and any one of them can be used in the object of the present invention.

Commercially available filling vessels are usable, and, for example, Nunc cryotube (Thermo Fisher Scientific), Nalgene cryovial (Thermo Fisher Scientific), Bi. File jacket tube (F·C·R & Bio Co., Ltd.), Freeze Bag (Nipro Corporation), Freeze Tube (Nipro Corporation), Terumo Syringe (Terumo Corporation), Nipro Syringe (Nipro Corporation), and the like are usable.

The capacity of the filling vessel is not particularly limited, and it is sufficient that the filling with a sufficient volume of a stock solution in which cells are suspended can be achieved. For example, the lower limit can be set to 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, or 2.0 mL, and the upper limit can be set to 1000 mL, 500 mL, 100 mL, 50 mL, 20 mL, 10 mL, or 5 mL. In this specification, the “capacity” of the filling vessel means “internal capacity”, and, in the case of commercially available products, does not mean the appropriate volume indicated by a manufacturer (e.g., a product marketed as a “1 ml vial” is recommended to be filled with up to 1 ml of liquid), but means the maximum volume in which a liquid can be preserved in such a sealed state that the filling vessel is actually filled with the liquid and sealed, for example, with a lid (in the case of a syringe, it is sufficient that the inside can be held with a plunger).

The ratio of the volume of the cell aggregate suspension to the capacity of the filling vessel is not particularly limited insofar as it is 60% or higher, and can be set to more preferably 65% or higher, more preferably 70% or higher, more preferably 75% or higher, more preferably 80% or higher, more preferably 85% or higher, and more preferably 90% or higher. The more preferable range varies depending on the type of cells constituting the target cell aggregate and the shape of the filling vessel used: for example, in the case of a cell aggregate composed of pluripotent stem cell-derived differentiated cells or somatic stem cells, the above-described ratio is preferably 80% or higher and particularly preferably 85% or higher.

As usual, when cells are preserved in the filling vessel, a certain amount of air is preferably introduced into the filling vessel irrespective of a non-frozen state or a frozen state. Surprisingly, it has been first confirmed in the present invention that, when a cell aggregate suspension is preserved in a non-frozen state, the ratio of the volume of the cell aggregate suspension to the capacity of the filling vessel is preferably high, and, by filling the cell aggregate suspension in the ratio ranges above, the cell aggregate suspension can be preserved and transported while maintaining the form of the cell aggregate.

When the filling vessel is a substantially cylindrical or rectangular parallelepiped and the filling vessel is divided in half such that the cross section is substantially rectangular and the area of the rectangular cross section is the largest, the value obtained by dividing the long side of the cross section by the short side can be set to preferably 0.8 or higher, more preferably 1.0 or higher, more preferably 1.2 or higher, more preferably 1.4 or higher, more preferably 1.6 or higher, more preferably 1.8 or higher, 2 or higher, more preferably 3 or higher, more preferably 4 or higher, more preferably 5 or higher, more preferably 6 or higher, more preferably 7 or higher, more preferably 8 or higher, more preferably 9 or higher, more preferably 10 or higher, more preferably 12 or higher, more preferably 15 or higher, or more preferably 20 or higher.

When the cross section cannot be said to be substantially a rectangular, e.g., the bottom of the filling vessel is hemispherical or V-shaped or the mouth of the filling vessel is narrow, the short side can be replaced with the inner diameter of the vessel, and the long side can be replaced with the height of a filling part. By the use of the filling vessel in the above-described ranges above, a cell aggregate suspension can be preserved and transported while maintaining the form of the cell aggregate.

When the filling vessel is a common vial or tubular, the inner diameter is preferably small. The preferable specific range varies depending on the capacity of the filling vessel, and therefore cannot be generalized. When the capacity is 0.8 to 10 ml, the ranges are 2.0 cm or lower, 1.5 cm or lower, 1.2 cm or lower, 1.0 cm or lower, and 0.8 cm or lower.

For the cell aggregate used in this step, pluripotent stem cells, pluripotent stem cell-derived differentiated cells, or somatic stem cells are used. As the cells, those described in the section “Cells” in “1-1. Definition of Terms” above are preferably used.

A method of producing a cell aggregate includes culturing pluripotent stem cells, pluripotent stem cell-derived differentiated cells, or somatic stem cells in a single-cell state serving as the source of a cell aggregate to cause the cells to aggregate. A culture method of producing the aggregates is not particularly limited, and the cell aggregate can be suitably produced by the suspension culture or the adherent culture.

A method of suspending a cell aggregate in a non-freezing stock solution is not particularly limited. For example, the suspension can be achieved by aeration, circulating a liquid, or other mechanical stirring. Examples of specific methods include methods, such as pipetting and tapping.

The non-freezing stock solution, in which a cell aggregate is suspended, is a solution containing a sodium salt, a potassium salt, and a calcium salt as described in the section “Cell aggregate suspension” in “1-1. Definition of Terms” above, and one that can preserve a cell aggregate in a non-frozen state may be used.

The types of the sodium salt, the potassium salt, and the calcium salt used in the present invention are not particularly limited. Each salt may contain one type of salt or a plurality of types of salts. Specific examples of each salt are as described above in Definition of Terms, but are not limited to the above.

As the salt contained in the stock solution used in the present invention, chloride can be further contained. Chloride may be contained as a salt that is not any of a sodium salt, a potassium salt, or a calcium salt, one or more of those salts may contain chloride, or all of the salts may contain chloride. For example, any one or more of a sodium salt, a potassium salt, and a calcium salt may be chloride. Specific examples of chloride are as described above in Definition of Terms, but are not limited to the above.

The concentrations of the sodium salt and the chloride contained in the stock solution used in the present invention are not particularly limited, and, for example, are individually 30 mM or higher, 35 mM or higher, 40 mM or higher, 45 mM or higher, 50 mM or higher, 60 mM or higher, 70 mM or higher, 75 mM or higher, 77 mM or higher, 80 mM or higher, 85 mM or higher, 90 mM or higher, 100 mM or higher, 109 mM or higher, 110 mM or higher, 115 mM or higher, 120 mM or higher, 125 mM or higher, 126 mM or higher, 127 mM or higher, 128 mM or higher, 129 mM or higher, or 130 mM or higher.

The upper limit of the concentration of the sodium salt is, for example, 160 mM or lower, 155 mM or lower, 154 mM or lower, 150 mM or lower, or 147.5 mM or lower. The upper limit of the concentration of the chloride is, for example, 180 mM or lower, 170 mM or lower, 165 mM or lower, or 160 mM or lower. The electrolyte concentration of the sodium salt and the chloride in the solution can be calculated in terms of 1 mM=1 mEq/L.

The concentration of the potassium salt contained in the non-freezing stock solution used in the present invention is not particularly limited, and is, for example, 0 mM or higher, 0.5 mM or higher, 1 mM or higher, 1.5 mM or higher, 2 mM or higher, 2.5 mM or higher, 3 mM or higher, or 3.5 mM or higher. The upper limit of the concentration of the potassium salt is, for example, 40 mM or lower, 35 mM or lower, 30 mM or lower, 25 mM or lower, 20 mM or lower, 15 mM or lower, 10 mM or lower, 8 mM or lower, 7.5 mM or lower, 7 mM or lower, 6 mM or lower, or 5 mM or lower. The electrolyte concentration of the potassium salt in the solution can be calculated in terms of 1 mM=1 mEq/L.

The concentration of the calcium salt contained in the non-freezing stock solution used in the present invention is not particularly limited, and is, for example, 0 mM or higher, 0.1 mM or higher, 0.5 mM or higher, or 1 mM or higher. The upper limit of the concentration of the calcium salt is, for example, 10 mM or lower, 8 mM or lower, 7 mM or lower, 6 mM or lower, 5 mM or lower, 4 mM or lower, 3 mM or lower, and 2.5 mM or lower. The electrolyte concentration of the calcium salt in the solution can be calculated in terms of 1 mM=2 mEq/L.

The concentration ratio of the sodium salt, the potassium salt, and the calcium salt contained in the non-freezing stock solution used in the present invention is not particularly limited. The electrolyte concentration of the sodium salt is, for example, 10 to 100, 20 to 90, 30 to 80, 35 to 70, 36 to 60, 36.5 to 50, 37 to 49, 40 to 48, 42 to 47, or 43 to 46.7, assuming that the electrolyte concentration of the calcium salt is 1.

The electrolyte concentration of the potassium salt is, for example, 0.1 to 10, 0.25 to 7.5, 0.5 to 7, 0.6 to 5, 0.7 to 4, 0.8 to 3, 0.875 to 2, 0.9 to 1.75, 1 to 1.6, 1.1 to 1.5, or 1.2 to 1.4, assuming that the electrolyte concentration of the calcium salt is 1.

The concentration ratio between the chloride and the sodium salt is not particularly limited, and for example, the concentration of chloride ions is 0.5 to 1.5, 0.75 to 1, 0.8 to 1.09, 0.81 to 1.08, 0.82 to 1.07, or 0.83 to 1.06, assuming that the sodium ion concentration is 1.

The pH of the non-freezing stock solution used in the present invention is not particularly limited insofar as the cells to be preserved survive. The specific pH is, for example, 3.5 to 8.5, 4 to 8, 4.5 to 7.5, or 5 to 7.5.

The osmotic pressure of the non-freezing stock solution used in the present invention is not particularly limited insofar as the cells to be preserved survive. For example, the non-freezing stock solution can be a hypotonic solution (less than 250 mOsm/L), an isotonic solution (250 to 380 mOsm/L) or a hypertonic solution (more than 380 mOsm/L). The specific osmotic pressure is, for example, 150 mOsm/L to 750 mOsm/L, 200 mOsm/L to 700 mOsm/L, 250 mOsm/L to 650 mOsm/L, 250 mOsm/L to 610 mOsm/L, 250 mOsm/L to 550 mOsm/L, 250 mOsm/L to 500 mOsm/L, 250 mOsm/L to 450 mOsm/L, 250 mOsm/L to 400 mOsm/L, 250 mOsm/L to 380 mOsm/L, 250 mOsm/L to 350 mOsm/L, 250 mOsm/L to 310 mOsm/L, 250 mOsm/L to 300 mOsm/L, 250 mOsm/L to 280 mOsm/L, or 250 mOsm/L to 270 mOsm/L. The osmotic pressure may be expressed as the osmotic pressure ratio to phycological saline (e.g., 306 mOsm/L).

The viscosity of the non-freezing stock solution used in the present invention is not particularly limited insofar as the cells to be preserved survive. For example, the viscosity is preferably 8 mPas or higher and preferably 18 mPas or lower at the temperature when the cells are suspended and/or preserved. The viscosity of the non-freezing stock solution can be measured, for example, using a TV-20 viscometer (Toki Sangyo Co., Ltd.) at a rotational speed of 10 rpm.

The non-freezing stock solution used in the present invention may additionally contain any other salt in addition to these salts. Specific examples thereof include lactate, acetate, carbonate, bicarbonate, phosphate, monohydrogen phosphate, dihydrogen phosphate, sulfate, citrate, gluconate, succinate, hydrochloride, nitrate, oxalate, borate, magnesium salt, zinc salt, hydrate, and the like.

It is preferable for the stock solution not to contain differentiation-inducing factors or active protein components that are considered to have a significant influence on the properties and characteristics, therapeutic efficacy, and the like of cells.

The non-freezing stock solution used in the present invention may be self-prepared or commercially available. Specific examples of components usable as the non-freezing stock solution of the present invention are as described above but are not limited thereto. For example, the Ringer's solution can be used as it is as the non-freezing stock solution or with the further addition of components described below to the Ringer's solution. However, the Ringer's solution is preferably used as it is. The Ringer's solution is typically a solution in which a sodium salt, a potassium salt, and a calcium salt all contain chloride, the osmotic pressure is 150 to 750 mOsm/L (e.g., 260 mOsm/L), the pH is 3.5 to 8.5, the electrolyte concentration of the sodium salt is 125 to 150 mEq/L, the electrolyte concentrations of the potassium salt and the calcium salt are individually 2.5 to 5 mEq/L, and the electrolyte concentration of the chloride is 105 to 160 mEq/L.

As the solution, a Ringer's basal solution, a Ringer's solution where the sodium salt concentration is higher than the chloride concentration, e.g., lactated Ringer's solution, acetated Ringer's solution, bicarbonated Ringer's solution, or a combination thereof, and those obtained by adding any additional components thereto, are usable. Examples of commercially available solutions include a Ringer's basal solution, such as Ringer's solution “Fuso” (Fuso Pharmaceutical Industries, Ltd. (for the composition and the like, see the attached document (No. 7) revised in March 2011), a lactated Ringer's solution, such as Nisori Injection (Pfizer Inc. (for the composition and the like, see the attached document (10th edition) revised in January 2013), Lactec D Injection (Otsuka Pharmaceutical Factory, Inc. (for the composition and the like, see the attached document (9th edition) revised in April 2012), or Sorbitol-Lactated Ringer's injection “FUSO” (Fuso Pharmaceutical Industries, Ltd. (for the composition and the like, see the attached document (12th edition) revised in April 2012), an acetated Ringer's solution, such as Veen (registered trademark)-D Injection (Fuso Pharmaceutical Industries, Ltd. (for the composition and the like, see the attached document (second edition) revised in August 2017) or Physio 140 Injection (Otsuka Pharmaceutical Factory, Inc. (for the composition and the like, see the attached document (9th edition) revised in April 2011), and a bicarbonated Ringer's solution, such as BICANATE (registered trademark) Injection (Otsuka Pharmaceutical Factory, Inc. (for the composition and the like, see the attached document (second edition) revised in April 2011) or a bicarbon Injection (Yoshindo Inc. (for the composition and the like, see the attached document (third edition) revised in October 2016).

To the non-freezing stock solution used in the present invention, a ROCK inhibitor is preferably added, particularly when the preservation target is a pluripotent stem cell. The addition of a ROCK inhibitor can strengthen the binding of cells in a cell aggregate, thereby enhancing the stability and the survivability of the cell aggregate.

The upper limit of the concentration of the ROCK inhibitor is not particularly limited, and can be determined in accordance with, for example, the range in which the ROCK inhibitor does not cause cell death, the range in which the ROCK inhibitor does not cause a deviation from undifferentiation, the solubility of the ROCK inhibitor, or the like. For example, as the final concentration of the non-freezing stock solution, the upper limit can be set to 50 μM, 40 μM, 30 μM, 20 μM, or 10 μM.

The lower limit of the concentration of the ROCK inhibitor is not particularly limited, and can be determined in accordance with the concentration at which the effect of strengthening a cell aggregate is sufficiently exhibited. For example, when added as the final concentration of the non-freezing stock solution, the lower limit can be set to 0.1 μM, 1 μM, 2 μM, 3 μM, 5 μM, 7 μM, or 10 μM.

The non-freezing stock solution used in the present invention can further contain any saccharide. Specific examples thereof include glucose, sucrose, fructose, sorbitol, maltose, trehalose, mixed saccharides (e.g., GFX, and the like), or combinations thereof. However, in the non-freezing stock solution of the present invention, the content of a specific saccharide, e.g., trehalose, is preferably low. For example, the trehalose content is 1.5 (w/v) % or lower, 1 (w/v) % or lower, 0.5 (w/v) % or lower, 0.1 (w/v) % or lower, or 0.05 (w/v) % or lower, and a trehalose-free non-freezing stock solution is more preferable.

The non-freezing stock solution of the present invention may contain, as appropriate, additives, such as stabilizers (e.g., polyethylene glycol), buffers (e.g., phosphate buffer, sodium acetate buffer), amino acids (non-essential amino acids, such as glutamine, alanine, asparagine, serine, aspartic acid, cysteine, glutamic acid, glycine, proline, tyrosine, and niacin), vitamins (e.g., choline chloride, pantothenic acid, folic acid, nicotinamide, pyridoxal hydrochloride, riboflavin, thiamine hydrochloride, ascorbic acid, biotin, inositol, and the like), and human serum albumin, as necessary. However, it is preferable to use fewer types of the additives.

The non-freezing stock solution used in the present invention is sometimes a non-freezing stock solution of a cell aggregate as a cell preparation. Therefore, it is preferable that components harmful to a subject individual or cells are not contained or contained in an amount in which no harmful effects are exhibited. For example, only pharmaceutically acceptable components may be contained. Specifically, a cryoprotectant-free solution is preferable. For example, the Ringer's solution and Ringer-based solutions, such as a lactated Ringer's solution, are preferable because they are not pharmacologically problematic and can be administered directly to a living body.

A “cryoprotectant” refers to a compound having an action of suppressing the generation of ice crystals in cryopreservation. The cryoprotectant in this specification includes a freeze inhibitor and an antifreeze agent. Specific examples of the cryoprotectant include dimethyl sulfoxide (DMSO), glycerol, polyethylene glycol (PEG), ethylene glycol, trimethylene glycol, dimethylacetamide, polyvinylpyrrolidone, and the like. For example, the non-freezing stock solution of the present invention is preferably free from DMSO.

This step is desirably performed under aseptic conditions. The aseptic conditions can be achieved by any known method, and a method to be used is not particularly limited. Specifically, the aseptic condition can be achieved by, for example, a laminar flow cabinet, an isolator, an aseptic work room, an access restricted barrier system, or a combination thereof. To maintain the aseptic condition, all instruments used in a first aspect of the present invention, such as syringes, suction means, tubes, and the like, need to be sterilized. Sterilization treatment to be used is not particularly limited insofar as it is capable of killing or removing microorganisms in an object to be sterilized to the extent that the object of the present invention is achieved.

For example, chemical sterilization using ethanol, sodium hypochlorite, or the like, heat sterilization, such as high-pressure steam sterilization or dry heat sterilization, gas sterilization using ozone gas, ethylene oxide gas, plasma hydrogen peroxide gas, or the like, radiation sterilization using ultraviolet rays, gamma rays, or electron beams, sterilization filtration, and the like are usable. Sterilization also includes, for example, disinfection.

In general, as a sterilization method, an appropriate method and an appropriate condition can be selected according to the type of microorganism, the contamination state, and the properties and conditions of an object to be sterilized. For example, “Guidance on the Manufacture of Sterile Pharmaceutical Products Produced by Terminal Sterilization” and “Guidance on the Manufacture of Sterile Pharmaceutical Products by Aseptic Processing” provided by the Ministry of Health, Labor and Welfare of Japan, the “WHO good manufacturing practices for sterile pharmaceutical products” provided by the world health organization (WHO), and the like may be used as reference.

### 1-2-2. Preservation Step

A preservation step is a step of preserving a cell aggregate suspension of pluripotent stem cells, pluripotent stem cell-derived differentiated cells, or somatic stem cells in a non-frozen state. Insofar as a cell aggregate suspension is held in the temperature range of the present invention, not only the preservation in fixed facilities but a transportation process of a cell aggregate using refrigerated cars or refrigerated vessels can also be regarded as the preservation step of the present invention.

### (Cells)

As the cell aggregate suspension used in this step, the cell aggregate suspension described in the section “Cell aggregate suspension” in “1-1. Definition of Terms” above is preferably used. The size of the cell aggregate used in this step is not particularly limited, and, when observed under a microscope, the lower limit of the average diameter of the maximum width sizes in an observation image of cell aggregates in the same culture system can be set to 30 μm, 40 μm, 50 μm, 60 μm, 70 μm, 80 μm, 90 μm, or 100 μm, while the upper limit can be set to 600 μm, 500 μm, 400 μm, 300 μm, 250 μm, 200 μm, or 150 μm. The average diameter as used herein is a diameter determined by the volume average.

The cell aggregate in this range is preferable because it allows a stock solution to easily permeate evenly into internal cells, improving the preservation efficiency. The sizes of all the aggregates in a culture solution are not required to fall within the ranges above, and, for example, the volume average size or the number average size may fall within the ranges above. Among cell aggregate populations manufactured by this suspension culture step, cell aggregates are preferable which have a lower limit in terms of volume average size of 30%, 40%, 50%, 60%, 70%, 75%, 80%, 85%, 90%, 95%, 98%, or 100% within the size ranges above.

### (Cell Density)

It is sufficient that the density of cells suspended in the stock solution is not a density at which the quality, such as a survival rate, of the cells is not particularly reduced or a density at which excessive aggregation of cell aggregates is not caused. For example, the lower limit is preferably 0.05×10⁶ cells/mL, 0.08×10⁶ cells/mL, 0.1×10⁶ cells/mL, 0.2×10⁶ cells/mL, 0.3×10⁶ cells/mL, 0.4×10⁶ cells/mL, 0.5×10⁶ cells/mL, or 1.0×10⁶ cells/mL. The upper limit is preferably 200×10⁶ cells/mL, 150×10⁶ cells/mL, 100×10⁶ cells/mL, 90×10⁶ cells/mL, 80×10⁶ cells/mL, 70×10⁶ cells/mL, 60×10⁶ cells/mL, 50×10⁶ cells/mL, 40×10⁶ cells/mL, 30×10⁶ cells/mL, or 20×10⁶ cells/mL.

### (Temperature)

The temperature at which a cell aggregate is suspended in the stock solution for preservation is preferably low and a temperature at which the stock solution is not frozen. For example, even in a 0° C. or lower environment, a state where the freezing point depression effect caused by additives or the like is produced, so that the stock solution is not frozen is non-frozen preservation. Specifically, the range is, for example, from −3° C. to 25° C., and preferably from −3° C. to 15° C. Inhibiting the activity of cells at low temperatures makes it possible to suppress the deterioration of the quality, such as the survival rate, of the cells and unexpected changes in the properties and characteristics of the cells.

At this time, the low temperature is, for example, −3° C., −2° C., −1° C., 0° C., 1° C., 2° C., or 3° C. The upper limit is not particularly limited, and is preferably 25° C., 20° C., 15° C., 12° C., 10° C., 9° C., 8° C., 7° C., 6° C., 5° C., or 4° C. for the reasons described above. However, depending on the conditions, the preservation can be achieved at higher temperatures. As a method of achieving such a temperature is not particularly limited, and examples thereof include preservation on ice, preservation in a refrigerated vessel, preservation in a cool incubator, preservation in a refrigerator, preservation in other constant temperature devices, or the like.

### (State in Preservation)

The method of preserving a cell aggregate suspension of the present invention has such an excellent advantageous effect that the shape of a cell aggregate can be maintained against vibrations or shocks in preservation. Therefore, the advantageous effects of the present invention can be more effectively exhibited when the filled cell aggregate suspension is preserved in a preservation vessel not in a static state but in a state of flowing in the preservation vessel by shaking, rotating, or the like as the state in preservation. Therefore, it is preferable that, as the state in preservation, an external factor, such as shaking or rotating, or the movement of the preservation vessel is preferable. The movement distance in that case is not particularly limited, and is, for example, 1 km or longer, 10 km or longer, 20 km or longer, or 50 km or longer.

In the present invention, the preservation of a cell aggregate suspension is preferably preformed together with the transportation (including a transportation state). The transportation as used herein refers to moving a preservation vessel filled with a cell aggregate suspension from a place where the preservation vessel was filled with the cell aggregate suspension to another place by some means. The means is not particularly limited, and examples thereof include cars (including two-wheeled vehicles, four-wheeled vehicles, trucks, and the like), trains, airplanes, or the like. It is a matter of course that plural of these means may be combined and that there may be a static state, such as a rest, during movement. The proportion of the transportation state in a period of the preservation step of the present invention is not particularly limited, and, for example, the proportion of time may be 30% or higher, 40% or higher, 50% or higher, or 60% or higher.

### (Viscosity)

The viscosity of a cell aggregate suspension is not particularly limited, and, for example, to suppress cell sedimentation, the lower limit is preferably 1 mPa·sec, 2 mPa·sec, 3 mPa·sec, 4 mPa·sec, 5 mPa·sec, 6 mPa·sec, 7 mPa·sec, 8 mPa·sec, 9 mPa·sec, or 10 mPa·sec. The upper limit may be any viscosity that allows appropriate administration, and, for example, may be 35 mPa·sec, 30 mPa·sec, 25 mPa·sec, or 20 mPa·sec. A method of measuring the viscosity is not particularly limited, and, for example, the viscosity can be measured using a rotational viscometer TV-20 (manufactured by Toki Sangyo Co., Ltd.).

### (Preservation Period)

A period in which a cell aggregate suspension is preserved is not particularly limited, and, for example, the lower limit may be a period of time achieving the preservation for a period of time required to transport the cell aggregate, and is preferably 1 hour, 2 hours, 4 hours, 8 hours, 16 hours, or 24 hours. The upper limit may be, for example, a period of time in which a stock solution deteriorates and the quality of a cell aggregate decreases, and may be 30 days, 25 days, 20 days, 16 days, 15 days, 14 days, 10 days, 7 days, 6 days, 5 days, 4 days, or 3 days.

The cell aggregate preserved in this step can also be re-cultured after preservation, for example, when the cell aggregate is a pluripotent stem cell or a progenitor cell of any tissue. In the case of a differentiated cell, the cell aggregate may be used for the administration to a patient or the like, for example, after replacement with any liquid or may be used for the administration to a patient in the form of a stock solution without any replacement.

### (Packing)

A cell aggregate suspension may be preserved via a packing step of packing a filling vessel filled with a cell aggregate suspension into a constant temperature vessel preferably together with a cold insulator. The constant temperature vessel as used herein may be one capable of blocking the effect of the external temperature to some extent and does not necessarily have to be one that keeps the temperature inside a vessel constant. It is a matter of course that one capable of controlling the temperature in a predetermined range is more preferable. Alternatively, a low temperature transportation package or a cold insulator may be used as appropriate. To accurately administer a cell aggregate to a subject to whom the cell aggregate should be administered, the filling vessel can be given with cell information and/or information on a subject to whom the cell aggregate is to be administered.

The constant-temperature transportation package used in the present invention is a transportation package having heat insulating properties and heat retaining properties that allow the temperature inside of a vessel to be kept in a certain range for a certain period of time. Although not particularly limited, it is preferable to use a heat insulating vessel containing a heat insulating material as the constant-temperature transportation package. Examples of the constant-temperature transportation package can include, but are not limited to, those containing insulating materials, such as vacuum insulating materials, expanded polystyrene insulating materials, rigid polyurethane foam insulating materials, and glass wool insulating materials.

Examples of a particularly suitable constant-temperature transportation package can include TACPack (registered trademark) commercially available from Tamai Kasei Co., Ltd., va-Q-tec (registered trademark) commercially available from va-Q-tec AG, and Neoace (registered trademark) commercially available from Inoac Corporation. The above-described constant-temperature transportation package is a constant-temperature transportation package capable of keeping the temperature in a vessel constant. As described below, the temperature during transportation is kept in a predetermined range, and therefore, when the constant-temperature transportation package is used, one that meets the specifications for keeping the temperature during transportation in a predetermined range should be selected.

As the cold insulator, various kinds of cold insulators, such as bagged compositions containing water and a highly absorbent resin (e.g., sodium polyacrylate) as the main component, are commercially available, and any of the cold insulators can be used for the object of the present invention.

When a filling vessel is housed inside the constant-temperature transportation package, the filling vessel is preferably placed such that the longest side of the filling vessel is directed sideways. Cells are sedimented in a cell suspension, and therefore, when the filling vessel is housed in the constant-temperature transportation package with the longest side directed sideways, the bottom area when the cells are sedimented becomes larger than that when the filling vessel is housed with the longest side directed vertically, and thus cell deterioration can be minimized.

Although not essentially required, it is preferable to place a cushioning material in any excess space in the constant-temperature transportation package after the filling vessel is housed in the constant-temperature transportation package. This can reduce damage to cells due to shocks during transportation. Various materials can be used as the cushioning material, and air cushioning materials (air buffer material), foam cushioning materials, petit rolls, packing paper, and the like are usable.

When the cell aggregate suspension is preserved under a specific condition of the ratio of the volume of the cell aggregate suspension to the capacity of the filling vessel, the survival rate of the cell aggregate of after the preservation is preferably 70% or higher. The survival rate is more preferably 75% or higher, 80% or higher, 85% or higher, 90% or higher, or 95% or higher. In the condition ranges examined in Examples below, the cell survival rate was 95% or higher (Example 1).

Further, it has been found that a case where a cell aggregate suspension is preserved under a specific condition of the ratio of the volume of the cell aggregate suspension to the capacity of the filling vessel has another advantageous effect in which the shape of the cell aggregate can be maintained (Examples 1 and 2). For treatment using cells, a cell aggregate is sometimes used to enhance the therapeutic efficacy. However, depending on the preservation conditions, such as vibrations in transportation, spheroids collapse, so that the defined aggregation diameter cannot be maintained in some cases.

The use of the method of the present invention makes it possible to preserve a cell aggregate while maintaining not only the survival rate of the cell aggregate but the quality including the shape of the cell aggregate. An aggregate of pluripotent stem cells, such as iPS cells produced by the suspension culture method, is not administered as it is, and it is used as therapeutic cells after treatment for differentiation. When an institution for manufacturing iPS cells and an institution for preparing a cell preparation by differentiating iPS cells into therapeutic cells are different from each other, the method of the present invention can be adopted as a means for suitably transporting the cell aggregate of pluripotent stem cells serving as the raw material of the therapeutic cell.

### EXAMPLES

### Example 1. Simulated Transportation Experiment of Adipose-Derived MSC Spheroid Under Non-Frozen Conditions

### 1. Preparation of Spheroid Suspension

A cryopreserved human adipose-derived MSC was thawed, and then subjected to adherent culture at a seeding density of 1×10⁴ cells/cm² at 37° C. under a 5% CO₂ atmosphere using a MEMα (Life Technologies Japan Ltd.) added with 5% hPL (AventaCell), 0.1% gentamicin (TAKATA Pharmaceutical Co., Ltd.), and 0.1% amphotericin B (0.25 mg/mL) (Bristol-Myers Company) as a medium. On Day 2 of culture, cells were detached, washed, and collected as single cells. Next, the collected cells were seeded such that the volume of culture solution to be 6 mL and the cell density at the start of the culture was 6.5×10⁶ cells/well and subjected to suspension culture at 37° C. under a 5% CO₂ atmosphere with the same medium as that in the adherent culture using a 60 mm EZSPHERE (registered trademark) SP dish (AGC Technoglass Co., Ltd.) as a culture vessel. Three days later, the medium was separated by centrifugation and spheroids were collected.

The spheroids were washed, and then suspended in a non-freezing stock solution for cell preparation (lactated Ringer's solution+4 (w/v) % dextran+1.5 (w/v) % HSA) such that the concentration was 1×10⁶ cells/mL to prepare a spheroid suspension. The volume average particle size of the spheroids at this time was 115 μm. The cell count was based on the result of unicellularizing the spheroids sampled at the end of the culture with TrypLE (trademark) Select (Life technologies Japan Ltd.) and pipetting, and measuring the cells using a NucleoCounter NC-200.

### 2. Filling of Spheroid Suspension

The prepared human adipose MSC spheroid suspension was filled into a 1 mL vial (10 mm inner diameter, 33 mm height) at the following levels.
(Level 1) The 1 mL vial was filled with 1.90 mL of the spheroid suspension (filling rate of 100%).
(Level 2) The 1 mL vial was filled with 1.81 mL of the spheroid suspension (filling rate of 95%).
(Level 3) The 1 mL vial was filled with 1.71 mL of the spheroid suspension (filling rate of 90%).
(Level 4) The 1 mL vial was filled with 1.62 mL of the spheroid suspension (filling rate of 85%).
(Level 5) The 1 mL vial was filled with 1.52 mL of the spheroid suspension (filling rate of 80%).
(Level 6) The 1 mL vial was filled with 1.43 mL of the spheroid suspension (filling rate of 75%).
(Level 7) The 1 mL vial was filled with 1.33 mL of the spheroid suspension (filling rate of 70%).
(Level 8) The 1 mL vial was filled with 1.24 mL of the spheroid suspension (filling rate of 65%).
(Level 9) The 1 mL vial was filled with 1.14 mL of the spheroid suspension (filling rate of 60%).

### 3. Non-Frozen Preservation of Spheroid Suspension Under Vibration Conditions

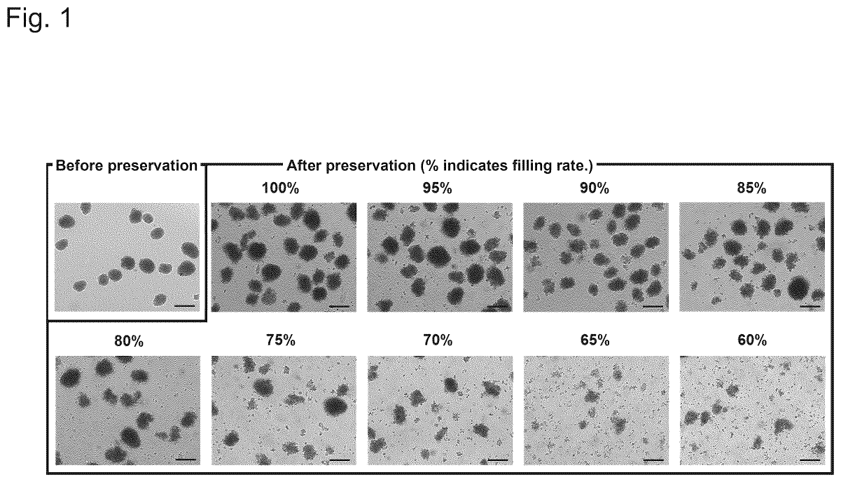

The vials at the levels after the filling were preserved for 24 hours in a 4° C. environment while being vibrated at 10 rpm using a mini-rotator (AS ONE Corporation.) to simulate the vibrations of a transportation environment. Thereafter, the quality was assessed by observing the survival rate (measured with a Nucleocounter NC-200) and the form of the cells in the suspension in each vessel. The survival rate of the cells at each level after the preservation is shown in Table 1 and the state of the suspension at each level after the preservation is illustrated in FIG. 1. The top left picture in FIG. 1 shows the state of the spheroids in the suspension at the start of the preservation, while the other nine pictures (Levels (1) to (9)) show the state of the spheroids in the suspension after the preservation.

As illustrated in FIG. 1, under the present conditions, most of the spheroids collapsed and did not maintain the shape under the conditions where the filling rate was 65% or lower (Levels (8) and (9)), and instead, a large number of single cells were observed. In contrast thereto, under the conditions where the filling rate is 70% or higher (Levels (1) to (7)), the spheroid shape is maintained, and, particularly when the filling rate is 80% or higher, surprisingly the collapse of the spheroids is hardly observed, and thus it was confirmed that the spheroids can be transported while maintaining the high quality in terms of both the survival rate and the shape maintenance.

TABLE 1

| | Survival rate (%) |
|---|---|
| Level (1) | 95 |
| Level (2) | 96 |
| Level (3) | 93 |
| Level (4) | 91 |
| Level (5) | 91 |
| Level (6) | 96 |
| Level (7) | 95 |
| Level (8) | 91 |
| Level (9) | 95 |

### Example 2. Simulated Transportation Experiment of Human iPS Cell Spheroid Under Non-Frozen Conditions

### 1. Preparation of Spheroid Suspension

Cryopreserved human iPS cells were thawed, and then seeded at 3000 cells/cm² on two 150 cm² dishes coated with iMatrix-511 (Nippi, Inc.) at 0.5 g/cm², and subjected to adherent culture at 37° C. under a 5% CO₂ atmosphere. As a medium, StemFit (registered trademark) AK02N (Ajinomoto Co., INC.) was used. The day of seeding of cells was defined as Day 0 of culture, and the complete medium exchange was performed on Days 1, 4, and 6 of culture. The medium amount was set to 60 mL/dish only in the medium exchange on Day 1 of culture, and 30 mL/dish otherwise. Y-27632 (Fujifilm Wako Pure Chemicals) was added to the medium only at the time of cell seeding such that the final concentration was 10 μM. On Day 7 of culture, the cells were detached and suspended in StemFit (registered trademark) AK02N (Ajinomoto Co., INC.) containing Y-27632 at a final concentration of 10 μM and IWR-1 endo (Fujifilm Wako Pure Chemicals) at a final concentration of 20 μM and collected as single cells.

Next, using a BioBlu 1c Single-Use Vessel (Eppendorf.com) was used as a culture vessel and a Bioflo (Eppendorf.com) as a reactor system for controlling the culture, cells were seeded such that the volume of culture solution to be 500 mL and the cell density at the start of culture was 5×10⁴ cells/mL, and the culture was started. As a medium at seeding, StemFit (registered trademark) AK02N (Ajinomoto Co., INC.) added with Y-27632 at a final concentration of 10 μM and IWR-1 endo at a final concentration of 20 μM was used. During the culture, surface aeration of the culture solution was performed while the culture temperature was kept at 37° C. and the gas supply amount was kept at 0.2 L/min. The carbon dioxide concentration in a supply gas was set to 5% at the start of the culture, and then reduced while adjusting the pH in the culture solution by up-and-down fluctuation using feedback control from the sensor values to maintain the pH at around 7.15 (suppressing a pH decrease). The supply gas was prepared by mixing any amount of carbon dioxide into air. The stirring speed was set to 87 rpm until Day 2 of culture and 79 rpm from Days 2 to 4 of culture.

The day when perfusion culture was performed to start culture was defined as Day 0 of culture. As a medium on Days 0 to 1 of culture, StemFit (registered trademark) AK02N (Ajinomoto Co., INC.) added with Y-27632 at a final concentration of 10 u was used. On Days 1 to 2 of culture, StemFit (registered trademark) AK02N (Ajinomoto Co., INC.) added with Y-27632 at a final concentration of 5 μM, IWR-1 endo at a final concentration of 20 μM, and LY333531 with a final concentration of 1 μM was used. On Days 2 to 4 of culture, StemFit (registered trademark) AK02N (Ajinomoto Co., INC.) added with Y-27632 at a final concentration of 2.5 M, IWR-1 endo with a final concentration of 20 μM, and LY333531 at a final concentration of 1 μM was used. Four days later, the medium was separated by centrifugation and spheroids were collected.

The spheroids were suspended in a Ringer's solution (Otsuka Pharmaceutical Industries) added with Y-27632 at a final concentration of 10 μM to prepare a spheroid suspension of 1×10⁶ cells/mL. The volume average particle size of the spheroids was 100 μm. The cell count was based on the result of unicellularizing the spheroids sampled at the end of the culture with Accutase (Innovative Cell Technology) and pipetting, and measuring the cells using a NucleoCounter NC-200.

### 2. Filling of Spheroid Suspension

The prepared human iPS cell spheroid suspension was filled into a 1 mL vial (10 mm inner diameter, 33 mm height) at the following levels.
(Level 10) The 1 mL vial was filled with 1.80 mL of the spheroid suspension (filling rate of 100%).
(Level 11) The 1 mL vial was filled with 1.62 mL of the spheroid suspension (filling rate of 90%).
(Level 12) The 1 mL vial was filled with 1.44 mL of the spheroid suspension (filling rate of 80%).
(Level 13) The 1 mL vial was filled with 1.26 mL of the spheroid suspension (filling rate of 70%).
(Level 14) The 1 mL vial was filled with 1.08 mL of the spheroid suspension (filling rate of 60%).
(Level 15) The 1 mL vial was filled with 0.9 mL of the spheroid suspension (filling rate of 50%).

### 3. Non-Frozen Preservation of Spheroid Suspension Under Vibration Conditions

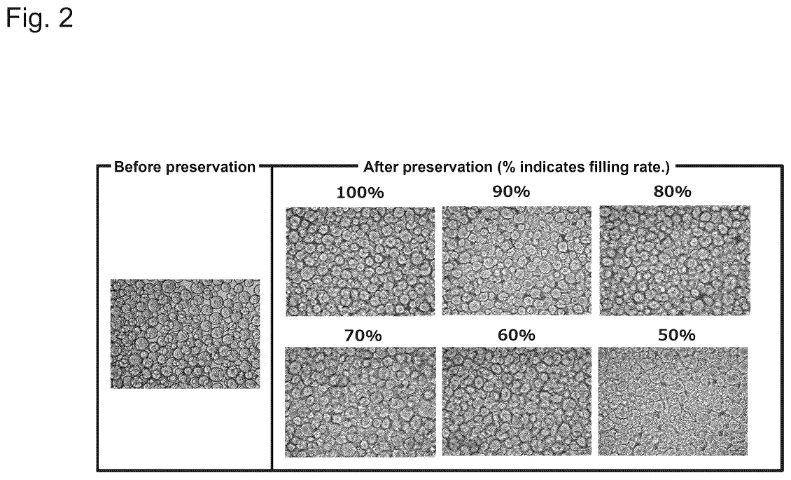

The vials at the levels after the filling were preserved for 24 hours in a 4° C. environment while being vibrated at 10 rpm using a mini-rotator (AS ONE Corporation.) to simulate the vibrations of a transportation environment. Thereafter, the quality was assessed by observing the form of the cells in each vessel. The state of the suspension at each level after the preservation is illustrated in FIG. 2. The top left picture in FIG. 2 shows the state of the spheroids in the suspension at the start of the preservation, while the other six pictures (Levels (10) to (15)) show the state of the spheroids in the suspension after the preservation. As illustrated in FIG. 2, under the condition where the filling rate is 50% (Level 15), most of the spheroids collapsed and instead, a large number of single cells were observed, which showed that there is a high possibility that the spheroid shape cannot be maintained by transportation, lowering the quality. In contrast thereto, under the conditions where the filling rate is 60% or higher (Levels (10) to (14)), surprisingly the collapse of the spheroids is hardly observed, and the volume average particle size of the spheroids after the preservation was measured to be 98.4 μm or higher in all the levels and most of the spheroids substantially maintained the particle size before the preservation, and thus it was confirmed that the spheroids can be transported while maintaining the high quality.

All publications, patents, and patent applications cited herein are incorporated herein by reference in their entirety.

1. A method of preserving a cell aggregate containing at least one of a pluripotent stem cell, a pluripotent stem cell-derived differentiated cell, and a somatic stem cell, the method comprising:
filling a filling vessel with a cell aggregate suspension such that a ratio of a volume of the cell aggregate suspension to a capacity of the filling vessel is 60% or higher; and
preserving the filling vessel in a non-frozen state for one hour or longer.

2. The method according to claim 1, wherein the ratio of the volume of the cell aggregate suspension to the capacity of the filling vessel is 70% or higher.

3. The method according to claim 1, wherein the ratio of the volume of the cell aggregate suspension to the capacity of the filling vessel is 80% or higher.

4. The method according to claim 1, wherein preserving the filling vessel comprises transporting the filling vessel.

5. The method according to claim 1, wherein the cell aggregate suspension is obtained by suspending a cell aggregate in a stock solution, the stock solution configured to allow preservation of a cell aggregate in the cell aggregate suspension in the non-frozen state.

6. The method according to claim 5, wherein the stock solution comprises a sodium salt.

7. The method according to claim 6, wherein the stock solution comprises a Ringer's solution or an aqueous solution prepared from a Ringer's solution.

8. The method according to claim 1, further comprising:
packing the filling vessel filled with the cell aggregate suspension in a constant temperature vessel together with a cold insulator,
wherein the filling vessel comprises at least one of cell information and information on a subject to which the cell aggregate suspension is to be administered.

9. The method according to claim 1, wherein a cell aggregate in the cell aggregate suspension to be preserved in the filling vessel has an average diameter of 600 μm or lower.

10. The method according to claim 1, wherein the filling vessel is preserved at 15° C. or lower.
